(19) United States
(12) Patent Application Publication
(10) Pub. No.: US 2025/0041249 A1
(43) Pub. Date: Feb. 6, 2025

(54) METHOD OF TREATING LIVER STEATOSIS WITH BIOAVAILABLE FORMULATION OF OLEOYLETHANOLAMIDE

(71) Applicant: THE REGENTS OF THE UNIVERSITY OF CALIFORNIA, OAKLAND, CA (US)

(72) Inventors: Daniele Piomelli, IRVINE, CA (US); Lin Lin, IRVINE, CA (US)

(73) Assignee: THE REGENTS OF THE UNIVERSITY OF CALIFORNIA, OAKLAND, CA (US)

(21) Appl. No.: 18/719,687

(22) PCT Filed: Dec. 12, 2022
(86) PCT No.: PCT/US2022/081372

§ 371 Date: Jun. 13, 2024

Related U.S. Application Data
(60) Provisional application No. 63/265,314, filed on Dec. 13, 2021.

(51) Int. Cl. A61K 31/16; A61K 9/00; A61K 9/107; A61K 47/14; A61K 47/24; A61K 47/44

(52) U.S. Cl. CPC A61K 31/16; A61K 9/0053; A61K 9/107; A61K 47/14; A61K 47/24; A61K 47/44

(57) ABSTRACT

Compositions and methods for delivery of oleoylethanolamide (OEA) in a bioavailable formulation that reduces lipid accumulation in the liver of a subject. Described herein is a novel OEA formulation that allows for compound vehiculation to the liver parenchyma, where this natural lipid amide exerts its anti-steatosis effects. This formulation provides a significantly greater oral bioavailability than either non-formulated OEA or a different formulation of OEA (Levagen®-OEA). Moreover, oral administration of the novel OEA formulation attenuates liver steatosis produced in a mouse model of human metabolic syndrome. The composition is useful in the treatment of human non-alcoholic liver steatosis.

This application claims benefit of U.S. provisional patent application No. 63/263,314, filed Dec. 13, 2021, the entire contents of which are incorporated by reference into this application.

### BACKGROUND

Nonalcoholic fatty liver disease (NAFLD) is the most common cause of chronic liver disease in the Western world. It is projected to become a leading indication for liver transplantation, superseding hepatitis C. NAFLD is associated with obesity and may progress to nonalcoholic steatohepatitis (NASH) and end-stage liver disease. There are no approved drug treatments for NAFLD or NASH. Animal and human studies suggest that oleoylethanolamide (OEA) might find application in the treatment of NAFLD, but the clinical use of this naturally occurring molecule is greatly limited by its poor bioavailability, due to a combination of unfavorable physicochemical properties and rapid enzyme-mediated degradation.

After a meal, the absorptive epithelium of the upper small intestine diverts a fraction of the oleic acid derived from the hydrolysis of dietary lipids toward the production of OEA (Schwartz et al., 2008, DiPatrizio and Piomelli, 2015). Acting as a local messenger within the gut, OEA reduces food intake through a mechanism that involves recruitment of peripheral sensory afferents (Rodriguez de Fonseca et al., 2001) and activation of central pathways that utilize oxytocin and histamine as neurotransmitters (Gaetani et al., 2010, Provensi et al., 2014). Several lines of evidence suggest that OEA initiates this response by engaging the ligand-operated transcription factor, peroxisome proliferator-activated receptor-a (PPAR-a). First, OEA is one of the most potent naturally occurring PPAR-α agonists identified to date (affinity constant, KD, ˜40 nM; median effective concentration, EC50, ≈120 nM) (Fu et al., 2003, Astarita et al., 2006a). Second, the satiety-inducing effects of OEA are abolished by PPAR-α deletion, are mimicked by synthetic PPAR-α agonists, and are associated with increased PPAR-a-regulated transcription in gut mucosa (Fu et al., 2003). Third, viral-mediated enhancement of OEA production in rat jejunum reduces food intake and concomitantly increases local expression of PPAR-α target genes (Fu et al., 2008). Finally, OEA levels in small intestine from various vertebrate species—including fish, snakes and rodents-rise from ≤50 nM in the fasting state to ≥250 nM after feeding (Astarita et al., 2006b, Fu et al., 2007, Tinoco et al., 2014, Igarashi et al., 2017), a concentration range that is compatible with PPAR-α activation (Fu et al., 2003, Astarita et al., 2006a). In sum, the available data indicate that OEA is a physiologically relevant endogenous agonist for small-intestinal PPAR-α, which participates in the control of satiety (DiPatrizio and Piomelli, 2015).

Feeding regulates OEA production also in the liver, but in an opposite direction to that seen in the gut: hepatic OEA levels rise in the fasting state and fall after feeding (Fu et al., 2007, Izzo et al., 2010). The molecular underpinnings and physiological significance of these changes have been investigated in detail. Studies have shown that fasting stimulates extra-hepatic mast cells to secrete histamine, which enters the liver via the portal circulation, activates G protein-coupled H1 receptors and triggers local OEA biosynthesis (Misto et al., 2018). Genetic or pharmacological manipulations that disrupt this process do not affect lipolysis, but markedly attenuate fasting-induced ketogenesis, thus revealing a previously unsuspected regulatory role for mast cell-derived histamine and liver OEA production in systemic lipid homeostasis (Misto et al., 2018). Consistent with such role, subchronic intraperitoneal administration of OEA attenuates liver steatosis in obese Zucker rats (Fu et al., 2005) and in a rat model of non-alcoholic fatty liver disease (Li et al., 2015). Moreover, intraperitoneal OEA administration stimulates fatty-acid oxidation in isolated rat hepatocytes and enhances ketone body production (ketogenesis) in live rats (Guzman et al., 2004). Finally, subchronic intraperitoneal OEA administration reduces lipid accumulation, inflammatory responses, and fibrosis in the liver of diet-induced obese mice (Lin et al., 2022; PMID: 35691287).

While intraperitoneal OEA administration is clearly beneficial in rodent models of liver steatosis, the clinical application of OEA as an oral treatment for NAFLD is hindered by two key factors: (1) following oral administration, OEA undergoes rapid in vivo degradation catalyzed by fatty acid amide hydrolase and other lipid amidases (Piomelli, 2013; PMID: 23567058); and (2) OEA is a lipophilic molecule with limited drug-like properties.

There remains a need for a means to deliver OEA in a manner that makes it bioavailable, particularly in a manner that makes it effective and available to the liver.

### SUMMARY

The compositions and methods described herein provide a means to deliver OEA in a bioavailable formulation that reduces lipid accumulation in the liver of a subject. Described herein is a novel OEA formulation that allows compound vehiculation to the liver parenchyma, where this natural lipid amide exerts its anti-steatosis effects. This formulation provides a significantly greater oral bioavailability than either non-formulated (‘neat’) OEA or a different formulation of OEA (Levagen®-OEA). Moreover, oral administration of the novel OEA formulation attenuates liver steatosis produced in a mouse model of human metabolic syndrome. The data described herein support use of the composition in the treatment of human NAFLD, including NASH.

Described herein is a composition comprising 5-15% by weight OEA and 85-95% self-emulsifying drug delivery system (SEDDS). In some embodiments, the SEDDS comprises a carrier oil comprising medium chain triglycerides, a citrus oil, and lecithin. In some embodiments, the composition promotes at least a twofold increase in bioavailability of the OEA. In some embodiments, the composition promotes at least a threefold increase in the bioavailability of the OEA. In some embodiments, the SEDDS comprises AquaCelle®.

The SEDDS marketed as AquaCelle® is described in U.S. Patent Publication No. 20190374494, published Dec. 12, 2019. See also Bremmell, K. E.; et al, A self-emulsifying Omega-3 ethyl ester formulation (AquaCelle) significantly improves eicosapentaenoic and docosahexaenoic acid bioavailability in healthy adults. Eur. J. Nutr. 2019, 59, 2729-2737. Further information about methods of delivering nutraceuticals can be found in Subramanian, P. Lipid-Based Nanocarrier System for the Effective Delivery of Nutraceuticals. Molecules 2021, 26, 5510 https://doi.org/10.3390/molecules26185510.

In some embodiments, the composition comprises 10-12% OEA and 88-90% AquaCelle®. In some embodiments, the composition comprises 11% OEA and 89% AquaCelle®. In some embodiments, the carrier oil comprises up to about 11% by weight, the citrus oil comprises up to about 10% by weight, and lecithin comprises up to about 11% by weight.

In some embodiments, the percentage composition of the SEDDS is such that, when dispersed in an aqueous environment, the composition forms a population of micelles with a mean diameter of about 1 to 20 micrometers. In some embodiments, the composition forms a population of micelles with a mean diameter of about 5 to 15 micrometers. In some embodiments, the composition forms a population of micelles with a mean diameter of about 12 to 13 micrometers.

In some embodiments, the composition further comprises an antioxidant. Representative antioxidants include, but are not limited to, lecithin, ascorbyl palmitate, d-alpha-tocopherol, dl-alpha-tocopherol, d-alpha-tocopheryl acetate, dl-alpha-tocopheryl acetate, d-alpha-tocopheryl acid succinate, dl-alpha-tocopheryl acid succinate, Vitamin E and derivatives thereof, Olive polyphenols, Algal polyphenols, and mixtures thereof. In some embodiments, the antioxidant is present at a concentration of 0-0.5% by weight. In some embodiments, the antioxidant is present at a concentration of about 0.01% by weight. In some embodiments, the antioxidant is present at a concentration of about 0.02% by weight. In some embodiments, the antioxidant is present at a concentration of about 0.1% by weight. In some embodiments, the antioxidant is present at a concentration of about 0.5% by weight.

In some embodiments, the composition further comprises an excipient. Representative excipients include, but are not limited to, colloidal silica, corn starch, hydroxypropylmethylcellulose (HPMC), maltodextrin, magnesium stearate, magnesium hydroxide, microcrystalline cellulose, dextrin, sorbitol, mannitol and trehalose. In some embodiments, the excipient is present at a concentration of about 30% to about 90% by weight. In some embodiments, the excipient is present at a concentration of about 30% to about 90% by weight.

Also described herein is a method of delivering oleoylethanolamide (OEA) to the liver of a subject in need thereof. In some embodiments, the method comprises oral administration of a composition as described herein. Also described is a method of reducing liver steatosis in a subject, as well as a method of treating non-alcoholic fatty liver disease (NAFLD) in a subject, and a method of treating non-alcoholic steatohepatitis (NASH) in a subject. In some embodiments, each of these methods comprises oral administration of a composition as described herein.

### BRIEF DESCRIPTION OF THE DRAWINGS

FIG. 1. Effects of HFD exposure on fasting-induced histamine signaling in portal circulation and the liver. (A, B) Histamine identification in portal plasma. (A) Representative LC/MS-MS tracing showing the elution of authentic [²H₄]-histamine and native histamine in portal plasma. (B) Mass spectrum of native and [²H₄]-histamine showing the presence of diagnostic ion [M−NH₃]⁺ (m/z=95.1 and m/z=99.1) in portal plasma. (C-E) Effects of free feeding (circles), food deprivation (squares), and food deprivation/refeeding (triangles) in chow-fed (empty symbols) and HFD-fed (filled symbols) mice. (C) Histamine concentrations in portal plasma. (D) Histamine H1 receptor (H1r) transcription in the liver. (E) Histamine H2 receptor (H2r) transcription in the liver. (F) Histamine H4 receptor (H4r) transcription in the liver. Data are expressed as mean±SEM (n=4-6) and were analyzed using two-way ANOVA followed by Šídák's multiple comparisons test. *p<0.05, **p<0.01, ***p<0.001.

FIG. 2. Effects of HFD exposure on fasting-induced OEA signaling in liver tissue. (A) Representative LC/MS-MS tracing showing the presence of OEA and its regional isomer VEA in the liver. (B-F) Effects of free feeding (circles), food deprivation (squares), and food deprivation/refeeding (triangles) in chow-fed (empty symbols) and HFD-fed (filled symbols) mice. (B) OEA content in the liver. (C) AEA content in the liver. (D) PEA content in the liver. (E) NAPE-PLD (NapepId) transcription in the liver. (F) FAAH (Faah) transcription in the liver. Data are expressed as mean±SEM (n=4-9) and were analyzed using two-way ANOVA followed by Šídák's multiple comparisons test. * or #p<0.05, ** or ##p<0.01, *** or ###p<0.001. VEA, vaccenoylethanolamide.

FIG. 3: Effects of VEH or OEA administration on liver of HFD-fed mice. (A) Representative images of the liver in situ (left) and liver sections stained with Oil Red O (2nd from the left), BODIPY (3rd from the left), and PSR (right). (B) Quantification of BODIPY fluorescence in livers from VEH-treated (empty symbols) or OEA-treated (filled symbols) mice exposed to HFD. (C) Quantification of PSR fluorescence in livers from VEH-treated (empty symbols) or OEA-treated (filled symbols) mice exposed to HFD. (D) Total triglyceride content in livers from VEH-treated (empty symbols) or OEA-treated (filled symbols) mice exposed to HFD. (E) Interleukin IL6 protein. (F-M) Liver transcription of Il1b (F), Ccl2 (G), Col1a1 (H), Hmox1 (I), Nrf1 (J), Tnfa (K), Tgfb1 (L), and Nqo1 (M). (B-G) Data are expressed as mean±SEM (n=4-6) and were analyzed using unpaired, two-tailed Student's t test. *p<0.05, **p<0.01, ***p<0.001. VEH, vehicle.

FIG. 4. Male C57BI/6J mice exposed to HFD develop obesity, hyperglycemia, and liver steatosis. (A) Time-course of body weight gain in mice exposed to regular chow (empty symbols) or HFD (filled symbols). (B) Fasting glucose concentrations in cardiac plasma from chow-fed or HFD-fed mice. (C) Total triglyceride content in liver of chow-fed or HFD-fed mice. (D) Representative images of liver tissue sections from chow-fed (left) or HFD-fed (right) mice stained with Oil Red O. Data are expressed as mean±S.E.M (A, n=20-26; B-C, n=4-7) and were analyzed using two-way ANOVA followed by Šídak's multiple comparisons test (A) or unpaired, two-tailed Student's t test (B,C). *P<0.05, **P<0.01. *** P<0.001.

FIG. 5. Effect of OEA administration on body weight gain of HFD-fed mice. (A) Body weight trajectory (A) before and (B) after the start of OEA treatment. Data are expressed as mean±S.E.M and were analyzed using two-way ANOVA followed by Šídak's multiple comparisons test. *P<0.05, **P<0.01, ***P<0.001.

FIG. 6. H&E staining of liver sections showing the effects of vehicle (VEH) or OEA on liver histology in diet-induced obese mice.

FIGS. 7A-7D. Pharmacokinetic (PK) profiles of non-formulated OEA (neat-OEA), Levagen®-OEA, and Aquacelle®-OEA in mice. Dosing corresponded to 30 mg/kg free OEA. Insets show area under the curve (AUC) values. (7A, 7B) Comparative PK profiles of neat-OEA versus Levagen®-OEA in (7A) plasma and (7B) liver. (7C, 7D) Comparative PK profiles of neat-OEA versus Aquacelle®-OEA in (7C) plasma and (7D) liver. Data are expressed as mean±SEM (n=9-11) and were analyzed using two-way ANOVA followed by Šídák's multiple comparisons test or unpaired t test, *P<0.05, **P<0.01, ***P<0.001.

FIG. 8. (A) Scheme summarizing the study design. (B) Body weight of all animal subjects included in the study after three weeks of high fat diet (HFD) exposure and before randomization into control (AquaCelle® alone, circles) and OEA-treated (AquaCelle®-OEA, triangles) groups. (C) Body weight gain in mice during treatment with AquaCelle® alone and AquaCelle®-OEA. Data are expressed as mean±SEM (n=9/group) and were analyzed using unpaired t test or two-way ANOVA followed by Šídak′s multiple comparisons test. *P<0.05, **P<0.01, ***P<0.001.

FIGS. 9A-9D. Effects of oral administration of AquaCelle® alone or AquaCelle®-OEA in diet-induced obese mice with liver steatosis. (9A) Representative images of excised livers (left), liver sections stained with BODIPY (middle), and liver sections stained with Oil Red O (right). (9B) Average liver weight in mice treated with AquaCelle® alone (circles) or AquaCelle®-OEA (triangles) (n=8/9). (9C) Quantification of BODIPY fluorescence in livers from mice treated with AquaCelle® alone or AquaCelle®-OEA (n=4/group). (9D) Droplet size distribution in liver sections from mice treated with AquaCelle® alone or AquaCelle®-OEA. Data are expressed as mean±SEM and were analyzed using unpaired, two-tailed Student's t test. *P<0.05, **P<0.01, ***P<0.001.

FIGS. 10A-10I. Plasma blood profile of diet-induced obese mice with liver steatosis treated with AquaCelle® alone or AquaCelle®-OEA. (10A) Triglycerides (TAG), (10B) AST, aspartate transaminase, (10C) ALT, alanine transaminase, (10D) cholesterol, (10E) glucose, (10F) albumin, (10G) globulin, (10H) amylase, (10I) CPK, creatine kinase. Veh, AquaCelle® alone; OEA, AquaCelle®-OEA, Data are expressed as mean±SEM (n=6/7) and were analyzed using unpaired, two-tailed Student's t test. *P<0.05, **P<0.01, ***P<0.001.

### DETAILED DESCRIPTION

The compositions and methods described herein provide a novel OEA formulation that allows vehiculation of the compound to the liver parenchyma, where this natural lipid amide exerts its anti-steatosis effects. This formulation provides a significantly greater oral bioavailability than either non-formulated OEA or Levagen®-OEA. Moreover, oral administration of the novel OEA formulation attenuates liver steatosis produced in a mouse model of human metabolic syndrome. The data described herein support use of the composition in the treatment of human non-alcoholic liver steatosis.

### Definitions

All scientific and technical terms used in this application have meanings commonly used in the art unless otherwise specified. As used in this application, the following words or phrases have the meanings specified.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant.

As used herein, “pharmaceutically acceptable carrier” or “excipient” includes any material which, when combined with an active ingredient, allows the ingredient to retain biological activity and is non-reactive with the subject's immune system. Compositions comprising such carriers are formulated by well-known conventional methods (see, for example, Remington's Pharmaceutical Sciences, 18th edition, A. Gennaro, ed., Mack Publishing Co., Easton, PA, 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### Compositions

Described herein is a composition comprising 5-15% by weight OEA and 85-95% self-emulsifying drug delivery system (SEDDS). In some embodiments, the SEDDS comprises a carrier oil comprising medium chain triglycerides, a citrus oil, and lecithin. In some embodiments, the composition promotes at least a twofold increase in bioavailability of the OEA when administered to a subject relative to administration of neat OEA. In some embodiments, the composition promotes at least a threefold increase in the bioavailability of the OEA. In some embodiments, the SEDDS comprises AquaCelle®.

The SEDDS marketed as AquaCelle® is described in U.S. Patent Publication No. 20190374494, published Dec. 12, 2019. See also Bremmell, K. E.; et al, A self-emulsifying Omega-3 ethyl ester formulation (AquaCelle®) significantly improves eicosapentaenoic and docosahexaenoic acid bioavailability in healthy adults. Eur. J. Nutr. 2019, 59, 2729-2737. Further information about methods of delivering nutraceuticals can be found in Subramanian, P. Lipid-Based Nanocarrier System for the Effective Delivery of Nutraceuticals. Molecules 2021, 26, 5510 https://doi.org/10.3390/molecules26185510.

In some embodiments, the composition comprises 10-12% OEA and 88-90% AquaCelle®. In some embodiments, the composition comprises 11% OEA and 89% AquaCelle®. In some embodiments, the carrier oil comprises up to about 11% by weight, the citrus oil comprises up to about 10% by weight, and lecithin comprises up to about 11% by weight.

In some embodiments, the percentage composition of the SEDDS is such that, when dispersed in an aqueous environment, the composition forms a population of micelles with a mean diameter of about 1 to 20 micrometers. In some embodiments, the composition forms a population of micelles with a mean diameter of about 5 to 15 micrometers. In some embodiments, the composition forms a population of micelles with a mean diameter of about 12 to 13 micrometers.

In some embodiments, the composition is formulated for delivery in an enteric coating, such as, for example, an enteric-coated capsule. Enteric coated capsules are designed to remain intact in the stomach and then to release the active substance in the intestine. Enteric coating can be applied to solid dosage forms, such as granules, pellets, capsules, or tablet, to improve drug bioavailability by preventing degradation of acid or gastric enzyme labile drugs.

In some embodiments, the composition further comprises an antioxidant. Representative antioxidants include, but are not limited to, lecithin, ascorbyl palmitate, d-alpha-tocopherol, dl-alpha-tocopherol, d-alpha-tocopheryl acetate, dl-alpha-tocopheryl acetate, d-alpha-tocopheryl acid succinate, dl-alpha-tocopheryl acid succinate, Vitamin E and derivatives thereof, Olive polyphenols, Algal polyphenols, and mixtures thereof. In some embodiments, the antioxidant is present at a concentration of 0-0.5% by weight. In some embodiments, the antioxidant is present at a concentration of about 0.01% by weight. In some embodiments, the antioxidant is present at a concentration of about 0.02% by weight. In some embodiments, the antioxidant is present at a concentration of about 0.1% by weight. In some embodiments, the antioxidant is present at a concentration of about 0.5% by weight.

In some embodiments, the composition further comprises an excipient. Representative excipients include, but are not limited to, colloidal silica, corn starch, hydroxypropylmethylcellulose (HPMC), maltodextrin, magnesium stearate, magnesium hydroxide, microcrystalline cellulose, dextrin, sorbitol, mannitol and trehalose. In some embodiments, the excipient is present at a concentration of about 30% to about 90% by weight. In some embodiments, the excipient is present at a concentration of about 30% to about 90% by weight.

### Methods

Provided is a method of delivering OEA to the liver of a subject in need thereof. In some embodiments, the method comprises oral administration of a composition as described herein. Also described is a method of reducing liver steatosis in a subject, as well as a method of treating NAFLD in a subject, and a method of treating NASH in a subject. In some embodiments, each of these methods comprises oral administration of a composition as described herein. As demonstrated in the examples below, administration of the composition results in reduced lipid accumulation in the liver, reduced circulating triglycerides, and reduced levels of the liver enzymes, AST and ALT.

In some embodiments, dosing is 30 to 90 mg per administration. In some embodiments, dosing is 90 to 300 mg. In yet other embodiments, dosing is 300 to 600 mg. The administering comprises providing a composition to a subject via a route known in the art, including but not limited to oral, buccal, rectal, or intragastric routes of administration. In certain embodiments, oral routes of administering a composition are preferred. The composition can be delivered alone or in combination with food or drink. In some embodiments, the composition is administered in an enteric coating, such as an enteric-coated capsule.

In some embodiments, the composition is administered daily. In some embodiments, administration is twice daily. In some embodiments, the composition is administered for four to six weeks. In some embodiments, the composition is administered for five weeks. In some embodiments, the composition is administered for 4-24 weeks.

### EXAMPLES

The following examples are presented to illustrate the present invention and to assist one of ordinary skill in making and using the same. The examples are not intended in any way to otherwise limit the scope of the invention.

### Example 1: Diet-Induced Obesity Disrupts Histamine-Dependent Oleoylethanolamide Signaling in Mouse Liver

This Example demonstrates that subchronic OEA administration reduces lipid accumulation and transcription of proinflammatory and profibrotic genes in liver of HFD-exposed mice. The data reported herein show that disruption of histamine-dependent OEA signaling in liver can contribute to pathology in obesity-associated non-alcoholic fatty liver disease.

Oleoylethanolamide (OEA) is an important lipid-derived regulator of energy balance [1]. In the small intestine, where its functions have been studied extensively, OEA is generated postprandially from diet-derived oleic acid [2] and acts as a local messenger to promote satiety [3-5]. This effect is mediated by the ligand-operated transcription factor, peroxisome proliferator-activated receptor-a (PPAR-α), which binds OEA with high affinity [6,7]. Food intake regulates OEA mobilization also in the liver, but in an opposite direction to that observed in small intestine: hepatic OEA content increases in the fasting state and decreases after feeding [8,9]. The physiological significance of these changes has been investigated in a recent study [10]. Its results suggest that fasting stimulates extrahepatic mast cells to release histamine, which enters the liver via the portal circulation and activates H1-type receptors to enhance local OEA production. Interventions that disrupt this signaling process reduce fasting-induced ketogenesis by approximately 50% [10]. This effect is consistent with the critical role played by PPAR-α in the control of ketogenesis as well as with data indicating that OEA engages PPAR-α to stimulate lipolysis, enhance fatty acid oxidation, and lower body weight gain and liver triacylglycerol content in obese rats and mice [12-15]. The findings summarized above delineate a paracrine signaling process that involves fasting-induced histamine release from mast cells into the portal circulation, stimulation of OEA formation in liver, and potentiation of PPAR-α-mediated ketogenesis by OEA. Here, we asked whether diet-induced obesity, which is known to disrupt feeding-dependent OEA production in the small intestine might affect such process.

### Materials and Methods

### Chemicals

We purchased OEA, palmitoylethanolamide (PEA), anandamide and their deuterium-containing analogs from Cayman Chemicals (Ann Arbor, MI). Vaccenoylethanolamide (VEA, 18:1 411) was prepared as described [16]. Histamine and [²H₄]-histamine were from Toronto Research Chemicals Canada (Toronto, CA), ethanolamine and Oil Red O from Sigma Aldrich (St. Louis, MO, USA) and BODIPY and ProLong™ Gold anti-fade mountant with DAPI (4′,6-diamidino-2-phenylindole) from Thermo Fisher Scientific (Waltham, MA, USA). All solvents and reagents were of the highest available grade.

### Animals

Male C57BI/6J mice (17 weeks) were purchased from Charles River (Wilmington, MA). They were group housed (4-5/cage) in ventilated cages with free access to food and water, unless indicated otherwise. They were maintained under a 12-h light/dark cycle (lights on at 6:30 am) at controlled temperature (22±1° C.) and relative humidity (55±10%) and were handled for 1 week before experiments. Housing, animal maintenance and all procedures were conducted in accordance with the National Institutes of Health Guidelines for the Care and Use of Laboratory Animals and were approved by the Institutional Animal Care and Use Committee of the University of California, Irvine.

### Diet-Induced Obesity

18-week-old mice were randomized into two groups; one received standard chow (6.5% kcal fat, Envigo 2020X, Livermore, CA) and the other a high-fat diet (HFD, 60% kcal fat, D12492, Research Diets, New Brunswick, NJ). Both were available ad libitum for 11 weeks. Body weight and food intake were recorded three times per week. Animals from both groups were randomly assigned to three cohorts: a) free feeding, b) fasting (from 6:00 pm to 12:00 pm on the following day) and c) fasting/refeeding (2 hr access to food after fasting). Mice assigned to the fasting and fasting/refeeding groups were housed in bottom-wired cages to prevent coprophagia.

### OEA Preparation

OEA was dissolved in a vehicle of 95% saline/5% Tween80 (v/v) shortly before experiments. It was administered by intraperitoneal (i.p.) injection at 5 mg/kg in a volume of 10 ml/kg.

### Tissue Collection

Portal blood was collected from anesthetized mice using a 1-mL syringe fit with a 23G×1 needle (0.6 mm×25 mm) coated with potassium-EDTA (K2-EDTA). Samples were transferred to 0.5 mL polypropylene tubes coated with K2-EDTA and placed on ice. The tubes were centrifuged for 15 min at 490×g and 4° C., and plasma layers were collected and stored at −80° C. for further processing. Animals were euthanized by decapitation and livers were removed, snap-frozen in dry ice and stored at −80° C. until analyses.

### Liver Triglyceride Measurements

Liver triglycerides were quantified using a colorimetric assay kit (Cayman Chemicals). Briefly, ˜30 mg of right liver lobe were mixed with 0.2 mL of assay buffer and processed following manufacturer's instructions.

### Blood Glucose Measurements

Groups of chow-fed and HFD mice (n=7-8) were transferred into bottom-wired cages and fasted overnight. Tail blood glucose was measured using an Accu-Chek Aviva meter and test strips (Roche Diabetes Care, Indianapolis, IN).

### Other Biochemical Analyses

A comprehensive blood chemistry panel was performed at Antech Diagnostics (Irvine, CA).

### Histological Analyses

Oil Red O Staining: We embedded liver samples in a mold, cut them into 7-μm thick sections with a cryostat and fixed them with 4% paraformaldehyde (PFA) for 15 min. After fixation, we immersed the sections in phosphate-buffered saline (PBS) for 2 min, placed them in isopropanol (60%) for 2 min, and incubated them with Oil Red O (60% in isopropanol) for 30 min. Finally, we dipped the sections in 60% isopropanol 40% water and mounted them onto glass slides with 50% glycerol. Images were taken at 20× magnification.

BODIPY staining: Liver sections (7-μm thick) were immersed in PBS for 5 min, fixed with 4% PFA for 15 min, and washed with PBS. Each sample was circled with a hydrophobic pen and stained with BODIPY (boron dipyrromethene; 1/1000 dilution) for 30 min. Samples were dipped in PBS and mounted with anti-fade mountant with DAPI (CAT: P36931). Images were captured at 20× magnification.

### Lipid Analyses

### Analyte Extraction

Frozen liver samples (˜30-40 mg) were transferred into 2 mL Precellys® soft tissue vials and diluted with ice-cold acetone (1 mL) containing internal standards ([²H₄]-OEA, [²H₄]-PEA and [²H₄]-anandamide, 100 nM each). Samples were homogenized for 1 min using a Bertin homogenizer at 4° C. in 15s/cycle for 2 cycles with 20 s pause between cycles. The homogenates were centrifuged for 15 min at 490×g at 4° C. and supernatants were transferred into 8-mL glass vials and dried under N₂. Each sample was diluted with chloroform/methanol/water (2 mL/1 mL/1 mL, vol), vortexed and centrifuged at 490×g for 15 min at 4° C. The organic phases were collected and dried under N₂. The pellets were reconstituted in acetonitrile (100 μL), transferred to deactivated glass inserts and placed inside amber glass vials for liquid chromatography-mass spectrometry (LC/MS-MS) analyses. For histamine extraction, we transferred portal plasma samples (50 μL) to 1.5-mL plastic vials and precipitated proteins by addition of ice-cold acetonitrile (0.4 mL) containing [²H₄]-histamine (50 μL; 0.5 μg/ml). [²H₄]-Histamine was prepared in a solution of distilled water and EDTA to prevent glass binding [17]. Samples were vortexed for 30 s and centrifuged for 10 min at 490×g and 4° C. Supernatants were transferred to deactivated glass inserts and placed inside amber glass vials for LC/MS-MS analyses.

### LC/MS-MS Analyses

OEA quantification: Fatty acyl ethanolamides were fractionated using a 1260 series LC system (Agilent Technologies, Santa Clara, CA). A step gradient separation was performed on a Poroshell 120 column (1.9 μm, 2.1×100 mm; Agilent Technologies, Wilmington, DE) with a mobile phase consisting of 0.1% formic acid in water as solvent A and 0.1% formic acid in acetonitrile as solvent B. A linear gradient was used: 0.0-9.5 min 80% B; 9.51-11.0 min 95% B; and 11.1-15.50 min maintained at 55% B. Column temperature was maintained at 40° C. and autosampler temperature at 9° C. Injection volume was 2 μl, flow rate was 0.3 ml/min, and total analysis time was 15.5 min. The injection needle was washed in the autosampler port for 20 s before each injection, using a wash solution consisting of 10% acetone in water/methanol/isopropanol/acetonitrile (1:1:1:1, vol). The mass spectrometer was operated in the positive electrospray ionization mode. Analytes were quantified by multiple reaction monitoring using acquisition parameters shown in Table 1. Capillary and nozzle voltages were 3500 V and 300-500 V, respectively. Drying gas temperature was 300° C. with a flow of 10 L/min. Sheath gas temperature was 300° C. with a flow of 10 L/hr. Nebulizer pressure was set at 40 psi. We used the MassHunter software (Agilent Technologies) for instrument control, data acquisition and analysis.

TABLE 1
Mass Spectrometry Acquisition Parameters

| | Precursor | Product | Fragmentation | Collision |
|---|---|---|---|---|
| Analyte | ion (m/z) | ion (m/z) | voltage (V) | energy (V) |
| Anandamide | 348.3 | 62.2 | 132 | 14 |
| [²H]-Anandamide | 352.3 | 66.2 | 127 | 14 |
| Histamine | 112 | 95 | 112 | 13 |
| [²H]-Histamine | 116 | 99 | 108 | 13 |
| OEA | 326.3 | 62.2 | 148 | 14 |
| [²H]-OEA | 330.3 | 66.2 | 143 | 14 |
| VEA | 326.3 | 62.2 | 148 | 14 |
| PEA | 300.3 | 62.2 | 132 | 14 |
| [²H]-PEA | 304.3 | 66.2 | 127 | 13 |

Histamine quantification: We used a BEH Amide column (1.7 μm, 2.1×50 mm, Waters Corporation, Milford, MA). The mobile phase consisted of 40 mM ammonium formate, pH adjusted to 3.0 with formic acid as solvent A and 0.1% formic acid in acetonitrile as solvent B. A linear gradient was used: 0.0-1.5 min 75% B 1.51-2.5 min 65% B; and 2.51-5.50 min maintained at 75% B. Flow rate was 0.5 ml/min. Column temperature was maintained at 40° C. and auto-sampler temperature at 9° C. Injection volume was 2 μL. The mass spectrometer was operated in the positive mode. Quantifications were performed using the MRM transitions reported in Table 1. The capillary voltage was 2.8 kV. Source parameters were as follows: drying gas temperature was 230° C., with a flow of 9 L/min; nebulizer pressure was set at 30 psi; sheath gas temperature was 300° C. with a flow of 12 L/min; capillary voltage was set at 2000 V.

### Real Time PCR

We extracted total RNA from liver using TRIzol™ reagent (Thermo Fisher Scientific, Waltham, MS) and purified it with the PureLink™ RNA Mini Kit (Invitrogen, Waltham, MS) as directed by the supplier. Prior to purification, samples were passed through a gDNA Eliminator spin column (Qiagen, Germantown, CA). We quantified RNA using NanoDrop 2000/2000-c spectrophotometer (Thermo Fisher Scientific). cDNA was synthesized from 2 mg of total RNA using the High-Capacity cDNA RT Kit with RNase inhibitor (Applied BioSystems, Foster City, CA) following manufacturer's instructions. First-strand cDNA was amplified using TaqMan™ Universal PCR Master Mixture (Thermo Fisher Scientific) Real-time PCR reactions were performed in 96-well plates using the CFX96™ Real-Time System (Bio-Rad, Hercules, CA). The thermal cycling conditions were as follows: initial denaturation set at 95° C. for 10 min, followed by 45 cycles, where each cycle was performed at 95° C. for 30 s followed by 55° C. for 60 s. ΔCt values were calculated using the geometric mean of three different housekeeping genes, and the relative fold changes over control groups (chow-free feed) were calculated by the 2-44Ct method [18]. Real-time PCR primers and fluorogenic probes were purchased from Applied Biosystems (TaqMan (R) Gene Expression Assays, Foster City, CA). We used TaqMan gene expression assays for mouse Actb (Mm00607939_s1), Hprt (Mm00446968_m1), Gapdh (Mm99999915_g1), Tnfa (Mm00443258_m1), Il1b (Mm00434228_m1), Tgfb1 (Mm_01178820_m1), Ccl2 (Mm_00441242_m1) Hmox1 (Mm_00516005_m1), Col1a1 (Mm_00801666_g1), Nqo1 (Mm_01135606_m1), Nrf1 (Mm01135606_m1), Faah (Mm_00515684_m1), and NapepId (Mm_00724596_m1) (Applied Biosystems, Foster City, CA).

### Statistical Analyses

Data were analyzed using Graphpad Prism version 8.0 for Windows (GraphPad Software, San Diego, CA). Statistical significance was determined using the two-tailed student's t-test, one-way or two-way analysis of variance (ANOVA) with Bonferroni post hoc tests for multiple comparisons, as appropriate. Differences between groups were considered statistically significant at values of p<0.05. Results are expressed as mean±SEM.

### Results

High-Fat Diet Disrupts Fasting-Induced Histamine Release into the Hepatic Portal System

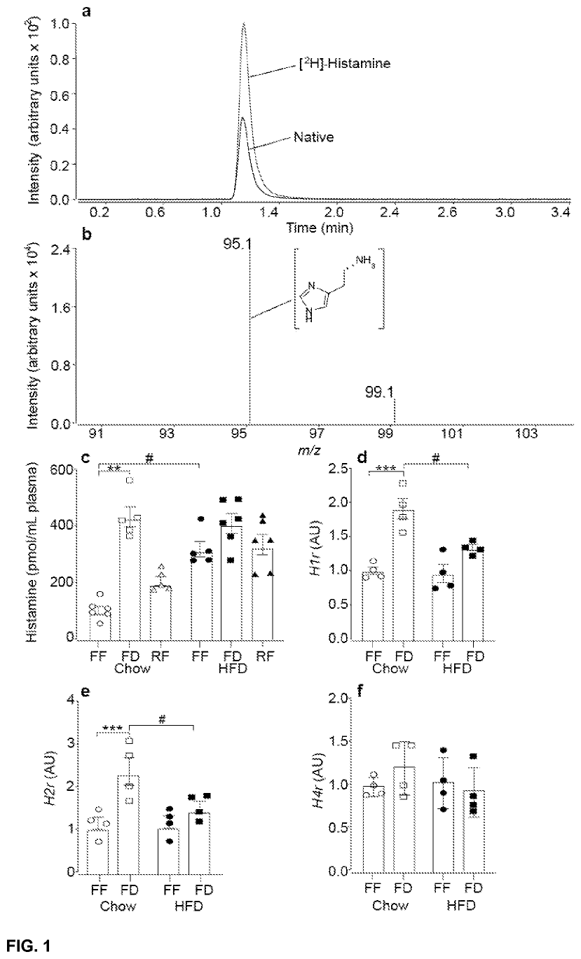

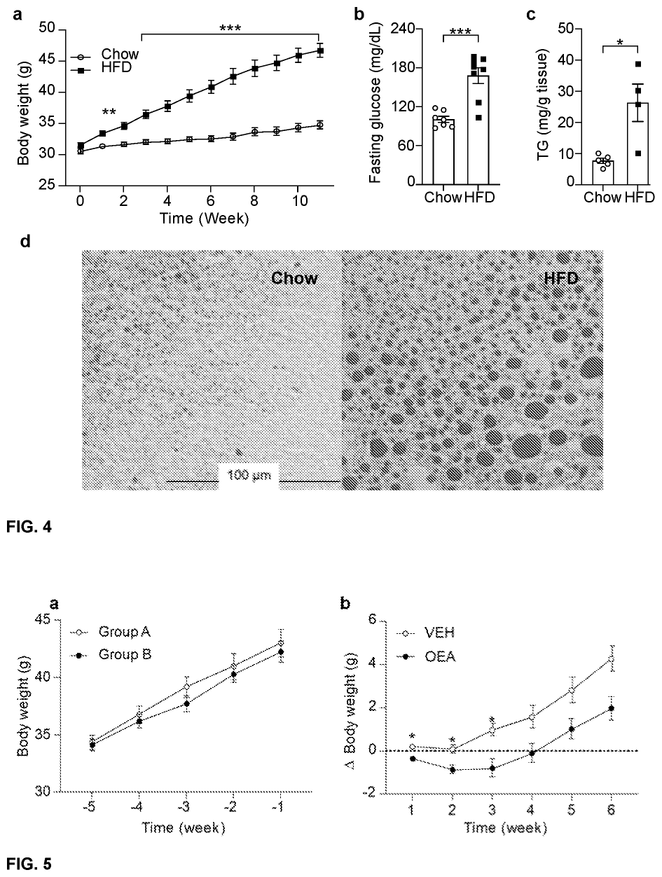

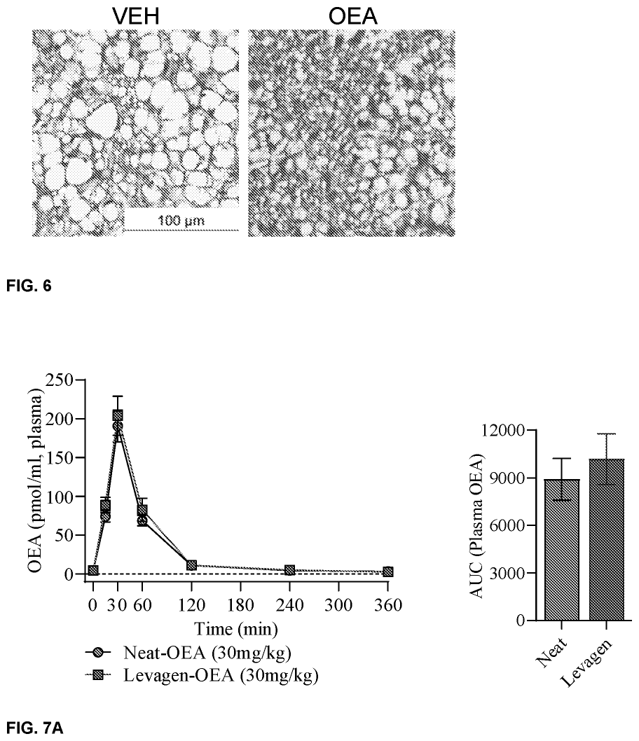

Fasting stimulates visceral mast cells to release histamine into the portal circulation [10]. To determine whether diet-induced obesity affects this response, we exposed mice for 12 weeks to HFD or regular chow (FIG. 4) and measured histamine concentrations in portal plasma using an LC-MS/MS assay developed ad hoc (FIG. 1a, b). Confirming previous results [10], in chow-fed mice food deprivation caused a substantial increase in portal histamine concentrations, which were 4 times higher in fasted than free-feeding mice and returned to baseline after 2-h refeeding (FIG. 1c). No such increase occurred in HFD-fed animals, whose free-feeding histamine levels were approximately 3 times higher than chow-fed controls and did not change in response to fasting (FIG. 1c). We also examined the impact of food deprivation on the transcription of H1 and H2 histamine receptors in liver of chow- and HFD-fed mice. As illustrated in FIGS. 1d and 1e, fasting caused a 2-fold elevation in the levels of H1 and H2 receptor mRNA in chow-fed controls, but had no effect in HFD-fed mice. The results suggest that exposure to HFD disrupts fasting-induced histamine release into the portal system as well as fasting-induced expression of histamine receptors in mouse liver.

### High-Fat Diet Disrupts Fasting-Induced OEA Production in Liver

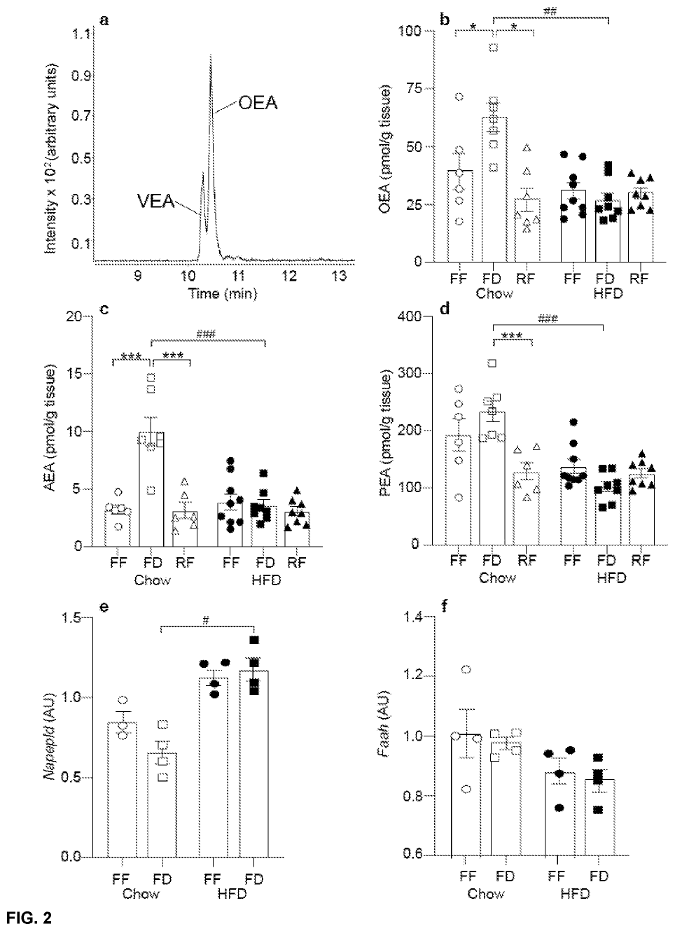

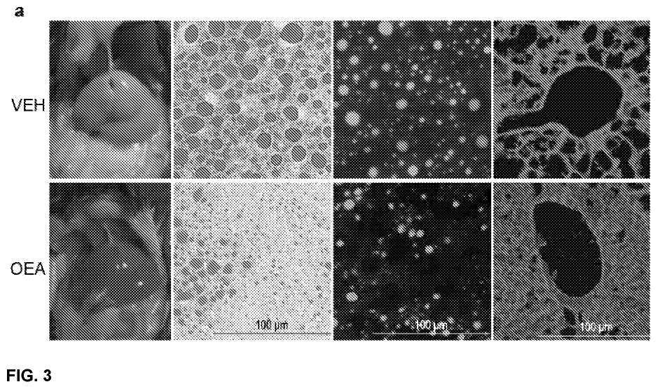

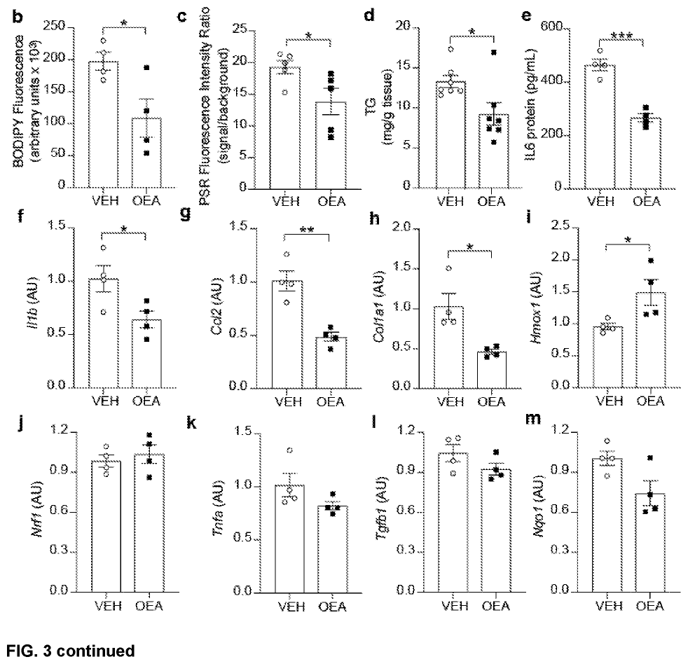

Mast cell-derived histamine stimulates OEA biosynthesis in liver through activation of H1 receptors [10]. To evaluate the impact of diet-induced obesity on this response, we measured OEA content in liver of chow- and HFD-fed mice using an LC-MS/MS assay that allows the separation of OEA (18:1 Δ⁹) from its regional isomer vaccenoylethanolamide (VEA, 18:1Δ¹¹) (FIG. 2a), which can interfere with OEA quantification [16, 19]. As previously reported [8,11], in chow-fed mice fasting caused a significant increase in liver OEA content, which returned to baseline after refeeding (FIG. 2b). No such response was observed in HFD-fed mice (FIG. 3b). Similarly, fasting increased liver levels of the endocannabinoid, anandamide, in chow-fed but not HFD-fed mice (FIG. 2b). The content of a third bioactive lipid amide, PEA, was not affected by food deprivation and was slightly decreased by HFD exposure (FIG. 2d). RT-PCR analyses revealed that transcription of NAPE-PLD (encoded in mice by the NapepId gene), which catalyzes OEA formation [20], was modestly increased by HFD while transcription of FAAH (Faah), which catalyzes OEA degradation [20], remained unaltered (FIG. 2e, f). Together, the findings indicate that HFD exposure disables fasting-induced OEA production in mouse liver.

### Exogenous OEA Corrects Lipid Accumulation in Liver of Obese Mice.

Diet-induced obesity is associated with the development of liver steatosis and fibrosis [21]. We asked whether OEA supplementation might correct these alterations in HFD-fed mice, which are defective in endogenous OEA production. We exposed mice to HFD for 5 weeks, randomized them into two groups, and treated them either with OEA (5 mg/kg, once daily) or vehicle for 6 additional weeks of HFD exposure. As expected [2, 12, 22-26], OEA administration reduced body weight gain and lowered circulating total cholesterol and liver transaminase levels (Table 2, FIG. 5). These effects were accompanied by improvement in hepatic lipid profile (FIG. 3). Oil Red O and BODIPY staining of liver sections showed that lipid content was reduced in OEA-treated mice compared to controls (FIG. 3a, b). Consistent with these results, liver triglyceride content was lower in OEA-treated than control mice (FIG. 3c). Further pointing to a protective action of OEA, we found that transcription of genes involved in liver inflammation and fibrosis-including C-C Motif Chemokine Ligand 2 (Ccl2) [27], interleukin 1B (1/1b) [28], and collagen type I a1 (Col1a1)-was attenuated in OEA-treated mice compared to controls (FIG. 3d-f), whereas transcription of the protective factor heme oxygenase 1 (Hmox1) was enhanced (FIG. 3g). Transcription of other relevant genes (Nrf1, Tnfa, Tgf1b, Nqo1) was not affected (FIG. 3h-j).

TABLE 2
Effects of vehicle (VEH) or OEA administration on body
weight gain and blood chemistry of HFD-fed mice.

| | Vehicle (n = 8) | OEA (n = 7) |
|---|---|---|
| A Body weight (g) | 4.26 ± 0.59 | 2.27 ± 0.5* |
| AST (SGOT) (IU/L) | 129.8 ± 11.6 | 86.1 ± 9.9* |
| ALT (SPGT) (IU/L) | 71.6 ± 7.5 | 45.4 ± 6.9* |
| Cholesterol (mg/dL) | 201.4 ± 5.9 | 173.6 ± 7.6* |
| Triglyceride (mg/dL) | 89.6 ± 6.1 | 81.0 ± 4.9 |
| Glucose (mg/dL) | 249.7 ± 12.1 | 250.5 ± 19.1 |

Abbreviations: AST: aspartate transaminase; ALT: alanine transaminase; SGOT: serum glutamic oxaloacetic transaminase; SGPT: Serum glutamic pyruvic transaminase.
Data are expressed as mean ± S.E.M and were analyzed using unpaired, two-tailed Student's t test.
*P < 0.05.

### Discussion

In fasting mice, histamine secreted from visceral mast cells into the portal system activates G_{q}-coupled H₁ receptors in the liver, stimulating local OEA biosynthesis [10]. Genetic or pharmacological manipulations that disable this process—including removal of mast cells, blockade of H₁ receptors, and deletion of histamine- or OEA-producing enzymes—reduce fasting-induced ketogenesis by approximately 50%. These findings identify histamine-dependent OEA signaling as a significant contributor to ketogenesis in mouse liver [10]. Here, we report that diet-induced obesity disrupts this paracrine signaling mechanism such that fasting no longer elicits histamine release and is unable of triggering hepatic OEA biosynthesis. Moreover, we provide new evidence confirming that OEA administration alleviates liver steatosis in obese rodents [13, 14, 22-24]. Similar results were reported in humans [25, 26]. Together, the present findings suggest that HFD-induced deficits in histamine release and liver OEA mobilization might contribute to the pathogenesis of non-alcoholic fatty liver disease (NAFLD), a condition that affects nearly 25% of adults worldwide [30].

The present results show that obesity is accompanied in mice by two notable changes in portal histamine (FIG. 1c). First, under free-feeding conditions, histamine levels were higher in portal blood of HFD-fed than chow-fed animals. Second, fasting-induced histamine secretion—a process that might be mediated by vagal inputs [31, 32]—was suppressed in HFD-exposed animals. The mechanism underlying these changes is unknown, but the proinflammatory environment generated in the gut by exposure to HFD is likely to be involved. Studies using the lymph fistula rat model have demonstrated that intestinal absorption of a fatty meal is accompanied by activation of mucosal mast cells and consequent release of histamine and prostaglandin D2 [33-35]. Intestinal mast cell degranulation was also documented in diet-induced obese mice [36]. It is thus plausible, though remains to be determined, that elevated histamine release in portal blood of obese free-feeding mice results from abnormal mast cell activity in inflamed mucosal tissue. Inflammation-related alterations in mast cell function might also explain the cells' inability to respond appropriately to fasting.

In addition to curbing histamine release from mast cells, HFD also blunted fasting-induced transcription of H₁ and H₂ receptors in liver (FIG. 1d, e), which may further impair the organ's ability to produce OEA. Guinea pig and rat hepatocytes express H₁ and H₂ receptors and, in vitro, their activation enhances glycogenolysis, gluconeogenesis, and ureagenesis [37, 38]. Consistent with those data, studies have shown that prolonged treatment with H₁ receptor antagonists and genetic deletion of H₂ receptors exacerbate hepatic steatosis in mice [39, 40].

Confirming previous work [13, 14], we found that subchronic treatment with OEA alleviates liver steatosis. In humans, obesity is a primary risk factor in the onset of NAFLD, which can progress to pathologies such as non-alcoholic steatohepatitis (NASH) and liver cirrhosis [41]. Interventions that reduce obesity slow down this progression but no pharmacological therapy is currently approved for either NAFLD or NASH [43]. Previous studies have suggested that OEA supplementation may improve prognosis in persons with NAFLD [24, 25] by activating PPAR-α in adipose organ and liver to enhance lipolysis [12, 44], possibly by enhancing fatty acid oxidation and ketogenesis [12, 44] and alleviating local inflammation [45-47]. The present results are consistent with that proposal and further identify histamine-dependent OEA signaling in liver as a possible pathogenic mechanism and target for therapeutic action.

### REFERENCES

DiPatrizio N V, Piomelli D. J Clin Invest. 2015; 125(3): 891-8.
Schwartz G J, et al. Cell Metab. 2008; 8(4): 281-8.
Gaetani S, Oveisi F, Piomelli D. Neuropsychopharmacology. 2003; 28(7): 1311-6.
Proulx K, et al. Am J Physiol Regul Integr Comp Physiol. 2005; 289(3): R729-37.
Rodriguez de Fonseca F, et al. Nature. 2001; 414(6860): 209-12.
Fu J, et al. Nature. 2003; 425(6953): 90-3.
Astarita G, et al. J Pharmacol Exp Ther. 2006; 318(2): 563-70.
Fu J, et al. J Biol Chem. 2007; 282(2): 1518-28.
Izzo A A, et al. Obesity (Silver Spring). 2010; 18(1): 55-62.
Misto A, et al. Cell Metab. 2019; 29(1): 91-102 e5.
Montagner A, et al. Gut. 2016; 65(7): 1202-14.
Guzman M, et al. J Biol Chem. 2004; 279(27): 27849-54.
Fu J, et al. Neuropharmacology. 2005; 48(8): 1147-53.
Li L, et al. J Pharmacol Sci. 2015; 127(3): 244-50.
Romano A, et al. Nutrients. 2021; 13(2).
Igarashi M, et al. Biochim Biophys Acta. 2015; 1851(9): 1218-26.
Verburg K M, et al. Agents Actions. 1984; 14(5-6): 633-6.
Livak K J, et al. Methods. 2001; 25(4): 402-8.
Rohrig W, et al. Anal Bioanal Chem. 2016; 408(22): 6141-51.
Piomelli D. Trends Endocrinol Metab. 2013; 24(7): 332-41.
Wang C Y, Liao J K. Methods Mol Biol. 2012; 821:421-33.
Giudetti A M, et al. Antioxidants (Basel). 2021; 10(8).
Romano A, et al. Nutrients. 2021; 13(2).
Hu J, et al. Front Pharmacol. 2020; 11:605065.
Tutunchi H, et al. Health Promot Perspect. 2020; 10(4): 373-82.
Tutunchi H, et al. J Cell Physiol. 2021; 236(1): 417-26.
Roh Y S, et al. Adv Exp Med Biol. 2018; 1061:45-53.
An S Y, et al. Front Pharmacol. 2021; 12:645703.
Ortiz C, et al. Curr Tissue Microenviron Rep. 2021:1-12.
Sayiner M, et al. Clin Liver Dis. 2016; 20(2): 205-14.
Stead R H, et al. Auton Neurosci. 2006; 125(1-2): 53-61.
Gottwald T P, et al. Neuroreport. 1995; 7(1): 313-7.
Zhou A, et al. Front Physiol. 2020; 11:4.
Ji Y, et al. Am J Physiol Gastrointest Liver Physiol. 2012; 302(11): G1292-300.
Ji Y, et al. Curr Opin Clin Nutr Metab Care. 2011; 14(4): 315-21.
Gu Y, et al. Int Arch Allergy Immunol. 2021:1-13.
Garcia-Sainz J A, et al. Mol Pharmacol. 1987; 31(3): 253-8.
Garcia-Sainz J A, et al. Eur J Pharmacol. 1992; 227(3): 325-31.
Raveendran V V, et al. Am J Physiol Gastrointest Liver Physiol. 2014; 307(2): G219-28.
Yamada S, et al. Pathol Int. 2016; 66(12): 661-71.
Sheka A C, et al. JAMA. 2020; 323(12): 1175-83.
Vilar-Gomez E, et al. Gastroenterology. 2015; 149(2): 367-78 e5; quiz e14-5.
Thiagarajan P, Aithal G P. J Clin Exp Hepatol. 2019; 9(4): 515-21.
Suarez J, et al. Dis Model Mech. 2014; 7(1): 129-41.
Montecucco F, et al. Thromb Haemost. 2015; 113(4): 838-50.
Payahoo L, et al. Adv Pharm Bull. 2018; 8(3): 479-87.
Lama A, et al. Biomed Pharmacother. 2020; 129:110368.

### Example 2: AquaCelle®-OEA has Greater Oral Bioavailability than Levagen®-OEA

This Example demonstrates that not all formulations designed to increase the bioavailability of lipids can be used interchangeably. As shown in this Example, AquaCelle®-OEA has a significantly greater oral bioavailability than either non-formulated (‘neat’) OEA or a different formulation of OEA (Levagen®-OEA).

### Materials and Methods

### Chemicals

Authentic oleoylethanolamide (OEA) and its deuterium-containing analog [²H₄]-OEA were obtained from Cayman Chemicals (Ann Arbor, MI). Three OEA formulations were tested: (1) non-formulated OEA (‘neat-OEA’); (2) OEA formulated with Levogen-Plus® technology (‘Levogen-OEA’); and (3) OEA formulated with AquaCelle® technology (‘AquaCelle-OEA’). All formulations were suspended in sterile saline/Tween-80 (95%/5%, vol/vol) shortly before experiments and were administered by gavage in a total volume of 10 mL/kg. Ethanolamine and Oil Red O were obtained from Sigma Aldrich (St. Louis, MO) and BODIPY and ProLong™ Gold anti-fade mountant with DAPI (4′,6-diamidino-2-phenylindole) from Thermo Fisher Scientific (Waltham, MA). All other reagents and analytical solvents were of the highest available grade.

### Animals

Male C57BI/6J mice (13 weeks of age) were purchased from Charles River (Wilmington, MA). They were group housed in ventilated cages (4-5 per cage) with free access to food (standard chow, 6.5% kcal fat, Envigo 2020X, Livermore, CA) and water, unless indicated otherwise. They were maintained under a 12-h light/dark cycle (lights on at 6:30 am) at controlled temperature (22±1° C.) and relative humidity (55±10%) and were handled for 1 week before experiments. Housing, animal maintenance, and all other procedures were conducted in accordance with the National Institutes of Health Guidelines for the Care and Use of Laboratory Animals and were approved by the Institutional Animal Care and Use Committee of the University of California, Irvine.

### Tissue Collection

Blood was collected by cardiac puncture into ethylenediamine delta-tetra-acetic acid (EDTA)-rinsed syringes and transferred into 1-mL polypropylene tubes containing spray-coated potassium-EDTA (K2-EDTA). Plasma was prepared by centrifugation at 1450×g at 40C for 15 min, and transferred into polypropylene tubes, which were immediately frozen and stored at −80° C. Animals were euthanized by decapitation, their livers were quickly harvested, the right lobe of each liver was separated from the other lobes, frozen on dry ice, and stored at −80° C. until analyses.

### Pharmacokinetics of OEA Formulations

We administered neat-OEA, Levagen-OEA, or AquaCelle-OEA to 18-week-old mice by gavage at a dose of 30 mg/kg. We anesthetized the animals with isoflurane at various time points after administration (in min=0, 15, 30, 45, 60, 120, 240, 360; n=9-11 mice per point) and processed blood and liver as described above.

### Blood Chemistry

A comprehensive blood chemistry panel was performed at Antech Diagnostics (Irvine, CA).

### Histological Analyses

Oil Red O Staining: We embedded liver samples (right lobe) in a mold, cut them with a cryostat into 7-μm thick sections, and fixed the sections with 4% paraformaldehyde (PFA) for 15 min. After fixation, we immersed the sections in phosphate-buffered saline (PBS) for 2 min, placed them in isopropanol (60%) for 2 min, and incubated them with Oil Red O (60% in isopropanol) for 30 min. Finally, we dipped the sections in 60% isopropanol 40% water and mounted them onto glass slides with 50% glycerol. Images were taken at 20× magnification [Lin et al. 2022].

BODIPY staining: Liver sections (7-μm thick) were immersed in PBS for 5 min, fixed with 4% PFA for 15 min, and washed with PBS. Each sample was circled with a hydrophobic pen and stained with BODIPY (1/1000 dilution, vol/vol) for 30 min. Samples were dipped in PBS and mounted with anti-fade mountant with DAPI (CAT: P36931). Images were captured at 20× magnification [Lin et al. 2022].

### Lipid Extraction

Frozen liver samples (˜30-40 mg) were transferred into 2 mL Precellys® soft tissue vials and diluted with ice-cold acetone (1 mL) containing internal standard ([²H₄]-OEA, 100 nM). Samples were homogenized for 1 min using a Bertin homogenizer at 4° C. in 15s/cycle for 2 cycles with 20 s pause between cycles. The homogenates were centrifuged for 15 min at 490×g at 4° C. and supernatants were transferred into 8-mL glass vials and dried under N2. Each sample was diluted with chloroform/methanol/water (2 mL/1 mL/1 mL, vol/vol), vortexed and centrifuged at 490×g for 15 min at 4° C. The organic phases were collected and dried under N2. The pellets were reconstituted in acetonitrile (100 μL), transferred to deactivated glass inserts and placed inside amber glass vials for liquid chromatography-mass spectrometry (LC/MS-MS) analyses.

### OEA Quantification

Chromatographic separations were carried out using a 1260 series LC system

(Agilent Technologies, Santa Clara, California) consisting of a binary pump, degasser, temperature-controlled autosampler, and column compartment, coupled to a 6460C triple quadrupole mass spectrometric detector with JetStream electrospray ionization (ESI) interface. A step gradient separation was performed on a Poroshell 120 column (1.9 μm, 2.1×100 mm; Agilent Technologies, Wilmington, DE) with a mobile phase consisting of 0.1% formic acid in water as solvent A and 0.1% formic acid in acetonitrile as solvent B. A linear gradient was used: 0.0-9.5 min 80% B; 9.51-11.0 min 95% B; and 11.1-15.50 min maintained at 55% B. Column temperature was maintained at 40° C. and autosampler temperature at 9° C. Injection volume was 2 μl, flow rate was 0.3 ml/min, and total analysis time was 15.5 min. The injection needle was washed in the autosampler port for 20 s before each injection, using a wash solution consisting of 10% acetone in water/methanol/isopropanol/acetonitrile (1:1:1:1, vol). The mass spectrometer was operated in the positive electrospray ionization mode, monitoring the following MRM transitions (m/z): OEA, 326.3>62.0; [2H4]-OEA, 330.3>66.0. For OEA fragmentation and collision voltages were set at 148 and 14 respectively and for [²H₄]-OEA they were 143 and 14 respectively. Capillary and nozzle voltages were 3500 V and 500 V, respectively. Drying gas temperature was 300° C. with a flow of 10 L/min. Sheath gas temperature was 300° C. with a flow of 10 L/hr. Nebulizer pressure was set at 40 psi. Lowest limit of quantification was 0.6 ng/ml (3.7 fmol/injection of 2.0 μL). We used the MassHunter software (Agilent Technologies, Santa Clara, CA) for instrument control, data acquisition and analysis.

### Statistical Analyses

Data were analyzed using Graphpad Prism version 8.0 for Windows (GraphPad Software, San Diego, CA). Statistical significance was determined using two-tailed Student's t test, one-way or two-way analysis of variance (ANOVA) with Bonferroni post hoc tests for multiple comparisons, as appropriate. Differences between groups were considered statistically significant at values of p<0.05. Results are expressed as mean±SEM.

### Results

### Pharmacokinetic Profile of AquaCelle®-OEA

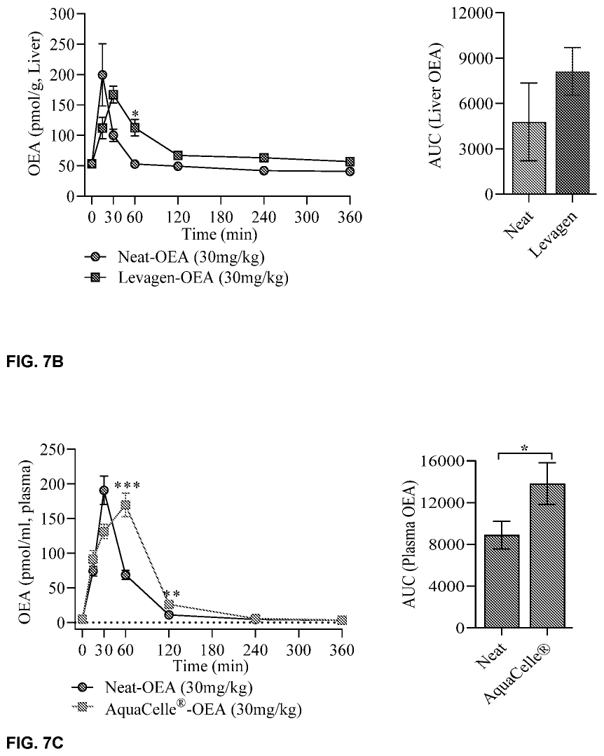

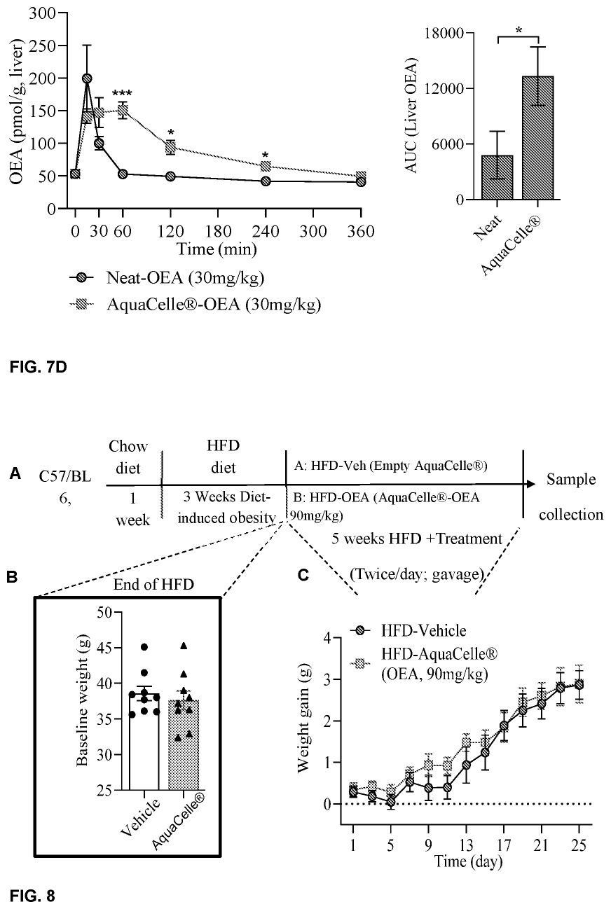

We compared the pharmacokinetic properties of three different OEA preparations-unformulated (‘neat’) OEA, Levogen®-OEA, and AquaCelle®-OEA-after a single oral (gavage) administration (equivalent to 30 mg per kg of free OEA) in male C57BI6 mice (n=X per group). The plasma OEA profile was similar in animals given neat OEA and Levogen®-OEA (FIG. 7A; insets show the area under the curve, AUC). We observed a small difference in liver OEA content, which was significantly higher at the 60-minute time point (P <0.05) in mice that received Levogen®-OEA compared to those given the unformulated compound (FIG. 7B). Nevertheless, AUC values were similar in the two groups (FIG. 7B). By contrast, significant differences were noted between neat OEA and AquaCelle®-OEA, with the latter producing significantly greater OEA levels both in plasma (FIG. 7C) and in liver (FIG. 7D). Indeed, the overall liver exposure to OEA (AUC) was approximately three times higher in mice treated with AquaCelle®-OEA than in those treated with unformulated compound (FIG. 7D). The results indicate that the AquaCelle® formulation technology effectively vehiculates orally administered OEA to the liver.

### Example 3: Treatment of Liver Steatosis with an Orally Bioavailable Formulation of Oleoylethanolamide

Systemic administration of the endogenous lipid-derived mediator, OEA, prevents lipid steatosis in diet-induced obese rats (PMID: 15910890) or in mutant mice lacking the fatty acid-binding protein, L-FABP (PMID: 22327204). The lipolytic properties of OEA were confirmed in the study described in Example 1, using a mouse model of diet-induced obesity. The main obstacle to the therapeutic application of these findings is the extremely poor bioavailability of OEA, which renders its use as an oral drug all but impossible. The company Gencor Pacific (https://www.gencorpacific.com) developed a self-emulsifying delivery system specifically designed to optimize the bioavailability of lipophilic molecules. This technology (AquaCelle®) achieves increased bioavailability by optimizing micelle formation and increasing the surface area of the oil-water interface. It consists of lipids, surfactants, co-surfactants and co-solvents that spontaneously form an emulsion in digestive fluids, aiding transportation through the intestinal epithelium.

This Example, together with Example 2, demonstrates that an AquaCelle®-OEA formulation (consisting of 11% OEA and 89% AquaCelle®) increases the overall oral bioavailability of OEA and, by doing so, enhances its therapeutic effects. The results show that repeated oral administration of AquaCelle®-OEA significantly attenuates hepatic lipid accumulation in a mouse model of diet-induced obesity. The findings suggest that AquaCelle®-OEA has a clinical application in the treatment of human non-alcoholic liver steatosis.

### Diet-Induced Obesity

We exposed mice (18 weeks old) to a high-fat diet (HFD, 60% kcal fat, D12492, Research Diets, New Brunswick, NJ) for 3 weeks. We randomized the animals into two groups and administered by gavage AquaCelle OEA (90 mg/kg) or its vehicle (AquaCelle without OEA) twice daily for 5 weeks, while continuing high-fat diet exposure. Individual body weight gain and cage food intake were recorded three times per week.

### Effects of AquaCelle®-OEA on Lipid Accumulation in Mouse Liver

A schematic overview of the study protocol is shown in FIG. 8A. Male C57BI6 mice were exposed to a high-fat diet (HFD, 60% kcal from fat) for three weeks and then randomized into two groups (n=9 per group) of approximately equal body weight (FIG. 8B). While continuing HFD exposure, the animals were given either AquaCelle®-OEA (equivalent to 90 mg per kg of free OEA) or AquaCelle® alone (vehicle) twice daily by oral gavage for five additional weeks. The mice were then euthanized, and their livers and cardiac blood were collected for analyses.

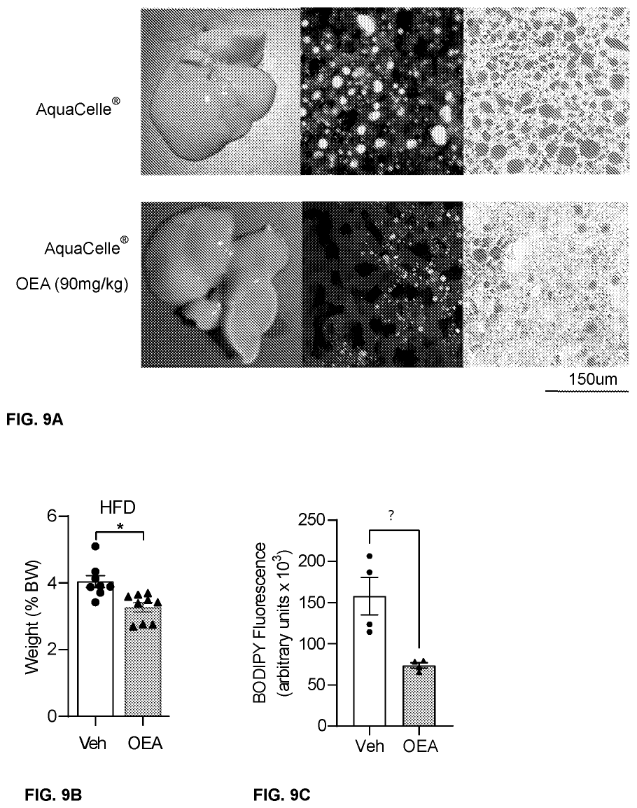

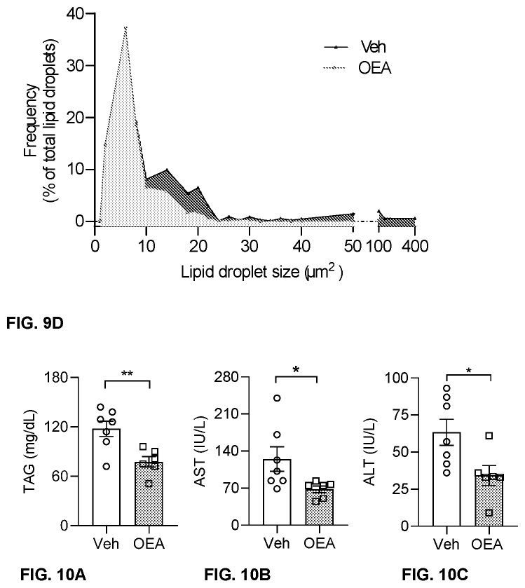

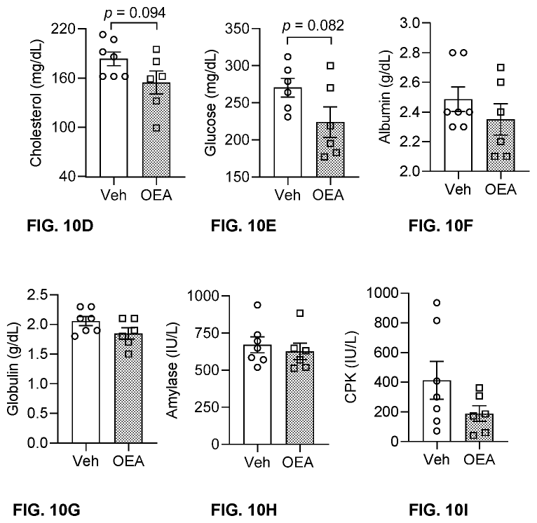

Administration of AquaCelle®-OEA had no effect on body-weight gain, which was similar in the two groups (FIG. 8C). Nevertheless, average liver weight was significantly lower (P<0.05; n=8=9) in mice treated with AquaCelle®-OEA than in mice treated with AquaCelle® alone (FIG. 9B). Moreover, the organ's external surface had a whiter appearance in the latter group (FIG. 9A, left). As both observations are consistent with an anti-steatosis effect of AquaCelle®-OEA, we measured lipid accumulation in sections of liver parenchyma using the intensively fluorescent lipophilic fluorophore, BODIPY®. The results show that administration of AquaCelle®-OEA significantly (P<0.05; n=4) reduced BODIPY® fluorescence in liver sections, compared to AquaCelle® alone (FIG. 9A, middle; FIG. 9C). The frequency distribution analysis illustrated in FIG. 9D further shows that treatment with AquaCelle®-OEA reduced both the average size of lipid droplets and their numbers. Staining with the lipophilic dye red Oil O confirmed these results (FIG. 9C, right). Together, the findings indicate that subchronic oral administration of AquaCelle®-OEA prevents lipid accumulation in liver of diet-induced obese mice without altering their body weight trajectory. Consistent with an improvement in hepatic steatosis, treatment with AquaCelle®-OEA lowered the circulating concentrations of triglycerides (P<0.01; n=7) (FIG. 10A) and of the liver enzymes, aspartate aminotransferase (AST) and alanine aminotransferase (ALT) (FIG. 10B, C). We found no statistically detectable effect on circulating total cholesterol, glucose, albumin, globulin, amylase, and creatine phosphokinase (CPK) (FIG. 10D-I).

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A composition comprising 5-15% by weight oleoylethanolamide (OEA) and 85-95% self-emulsifying drug delivery system (SEDDS), wherein the SEDDS comprises a carrier oil comprising medium chain triglycerides, a citrus oil, and lecithin, and wherein the composition promotes at least a twofold increase in bioavailability of the OEA.

2. The composition of claim 1, wherein the SEDDS comprises AquaCelle®.

3. The composition of claim 2, wherein the composition comprises 10-12% OEA and 88-90% AquaCelle®.

4. The composition of claim 2, wherein the composition comprises 11% OEA and 89% AquaCelle®.

5. A method of delivering oleoylethanolamide (OEA) to the liver of a subject in need thereof, the method comprising oral administration of a composition of claim 1.

6. A method of reducing liver steatosis in a subject, the method comprising oral administration of the composition of claim 1.

7. A method of treating non-alcoholic fatty liver disease (NAFLD) in a subject, the method comprising oral administration of the composition of claim 1.

8. A method of treating non-alcoholic steatohepatitis (NASH) in a subject, the method comprising oral administration of the composition of claim 1.

9. The method of claim 6, wherein the subject is human.

10. The method of claim 6, wherein the composition is administered in an enteric-coated capsule.

11. The method of claim 6, wherein the composition is administered once daily for four to 24 weeks.

12. The method of claim 11, wherein the composition is administered twice daily for four to 24 weeks.

13. The method of claim 7, wherein the subject is human.

14. The method of claim 7, wherein the composition is administered in an enteric-coated capsule.

15. The method of claim 7, wherein the composition is administered once daily for four to 24 weeks.

16. The method of claim 7, wherein the composition is administered twice daily for four to 24 weeks.

17. The method of claim 8, wherein the subject is human.

18. The method of claim 8, wherein the composition is administered in an enteric-coated capsule.

19. The method of claim 8, wherein the composition is administered once daily for four to 24 weeks.

20. The method of claim 8, wherein the composition is administered twice daily for four to 24 weeks.
